(19) United States
(12) Patent Application Publication
(10) Pub. No.: US 2005/0008587 A1
(43) Pub. Date: Jan. 13, 2005

(54) Cosmetic and dermatological photoprotective formulations with a content of hydroxybenzophenones, triazine derivatives and/or benzotriazole derivatives

(75) Inventors: Jens Schulz, Schenefeld (DE); Anja Goppel, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/840,430

(22) Filed: May 7, 2004

Related U.S. Application Data
(63) Continuation of application No. PCT/EP02/11783, filed on Oct. 22, 2002.

(30) Foreign Application Priority Data
Nov. 9, 2001 (DE) 101 55 963.1

(51) Int. Cl. A61K 7/42

(52) U.S. Cl. 424/59

(57) ABSTRACT

A photoprotective cosmetic or dermatological composition which comprises a triazine derivative and/or a benzotriazole derivative and also a hydroxybenzophenone.

### CROSS REFERENCE TO RELATED APPLICATIONS

The present application is a continuation of International Application No. PCT/EP02/11783, filed Oct. 22, 2002, the entire disclosure whereof is expressly incorporated by reference herein, which claims priority under 35 U.S.C.§ 119 of German Patent Application 101 55 963.1, filed Nov. 9, 2001.

### BACKGROUND OF THE INVENTION

1. Field of the Invention

The present invention relates to cosmetic and/or dermatological photoprotective preparations comprising hydroxybenzophenones besides triazine derivatives and/or benzotriazole derivatives.

2. Discussion of Background Information

The harmful effect of the ultraviolet part of solar radiation on the skin is generally known. Depending on their particular wavelength, the rays have different effects on the skin as an organ: UV-C radiation with a wavelength of less than 290 nm is absorbed by the ozone layer in the earth's atmosphere and therefore has no physiological significance. By contrast, rays in the range between 290 nm and 320 nm, the UV-B region, cause erythema, simple sunburn or even burns of greater or lesser severity. A maximum for the erythema activity of sunlight is given as the relatively narrow range around 308 nm.

Numerous compounds are known for protecting against UV-B radiation; these are, for example, derivatives of 3-benzylidenecamphor, 4-aminobenzoic acid, of cinnamic acid, of salicylic acid, and of benzophenone.

### SUMMARY OF THE INVENTION

It has for a long time been incorrectly assumed that the long-wave UV-A radiation with a wavelength between 320 nm and 400 nm has only a negligible biological effect. However, it has in the meantime been demonstrated by numerous studies that UV-A radiation is far more hazardous than UV-B radiation with regard to the triggering of photodynamic, specifically phototoxic, reactions and chronic changes in the skin. The harmful effect of UV-B radiation can also be further intensified by UV-A radiation.

Thus, it has been found, inter alia, that even UV-A radiation under entirely normal everyday conditions is sufficient to damage, within a short time, the collagen and elastin fibers which are of essential importance for the structure and strength of the skin. This results in chronic light-induced changes in the skin—the skin “ages” prematurely. The clinical appearance of skin aged by light includes, for example, wrinkles and lines and an irregular, furrowed relief. In addition, the areas affected by light-induced skin aging may have irregular pigmentation. The formation of brown spots, keratoses and even carcinomas or malignant melanomas is also possible. Skin aged prematurely by everyday UV exposure is additionally characterized by a lower activity of the Langerhans cells and slight, chronic inflammation.

Approximately 90% of the ultraviolet radiation which reaches the earth consists of UV-A rays. Whereas UV-B radiation varies greatly depending on numerous factors (e.g. season and time of day or latitude), UV-A radiation remains relatively constant from day to day irrespective of seasonal and diurnal or geographic factors. At the same time, most of the UV-A radiation penetrates into the living epidermis, while approximately 70% of the UV-B rays are retained by the horny layer.

It is therefore of fundamental importance that cosmetic and dermatological photoprotective preparations offer adequate protection both against UV-B radiation and also against UV-A radiation.

In general, the light absorption behavior of photoprotective filter substances is very well known and documented, especially since most industrialized countries have positive lists for the use of such substances, which impose very strict standards on the documentation.

However, the use concentration of known photoprotective filter substances which are sparingly soluble or in the form of a solid is often—especially in combination with other substances to be dissolved—restricted and gives rise to considerable formulation difficulties in achieving relatively high protection factors and/or UV-A protection performance.

### DETAILED DESCRIPTION OF THE PRESENT INVENTION

It was therefore the object of the present invention to formulate stable photoprotective preparations in which the solubility of the UV filters which are sparingly soluble or in the form of a solid is increased.

It was surprising and could not have been foreseen by the person skilled in the art that protective cosmetic and/or dermatological preparations, characterized in that they comprise
(a) at least one hydroxybenzophenone and
(b) at least one triazine derivative and/or benzotriazole derivative besides, where necessary, further cosmetic active ingredients, auxiliaries and additives, overcome the disadvantages of the prior art.

Besides one or more oil phases, the preparations for the purposes of the present invention can preferably additionally comprise one or more water phases and, for example, be in the form of W/O, O/W, W/O/W or O/W/O emulsions. Such formulations can preferably also be a microemulsion (e.g. a PIT emulsion), a solid emulsions (i.e. an emulsion which is stabilized by solids, e.g. a Pickering emulsion phase), a sprayable emulsion or a hydrodispersion. Furthermore, the preparations for the purposes of the present invention may also be virtually free from water (water content below 5% by weight based on the total weight of the preparation).

The preparations according to the invention are entirely satisfactory preparations in every respect which are not restricted to a limited choice of raw materials. Accordingly, they are particularly suitable for serving as a base for preparation forms with diverse end-use applications. The preparations according to the invention exhibit very good sensory and cosmetic properties, such as, for example, extensibility on the skin or the ability to absorb into the skin, and are further characterized by very good photoprotective effectiveness coupled with excellent skincare data.

The UV protection performance of sunscreens or of the UV filters on which they are based is generally determined in biological effectiveness tests under standardized conditions. For the purposes of the present invention “UV protection performance” means both the protection performance against UV-A radiation and also against UV-B radiation.

A measure of the UV protection performance are, for the purposes of the present invention, for example the sun protection factor (SPF) and also IPD values and the like.

The sun protection factor (SPF) gives the extension of solar irradiation permitted as a result of use of the sunscreen. It is the quotient of erythema threshold time with sunscreen and erythema threshold time without sunscreen.

To test the UV-A protection performance, use is usually made of the IPD method (IPD≡immediate pigment darkening). Similar to the determination of the sun protection factor, this method gives a value which indicates how much longer the skin protected with the sunscreen can be irradiated with UV-A radiation until the pigmentation which occurs is the same as for the unprotected skin.

Another test method which has become established throughout Europe is the Australian standard AS/NZS 2604: 1997. In this test, the absorption of the preparation in the UV-A region is measured. In order to satisfy this standard, the preparation must absorb at least 90% of the UV-A radiation in the range from 320 to 360 nm.

The hydroxybenzophenones according to the invention are characterized by the following structural formula: 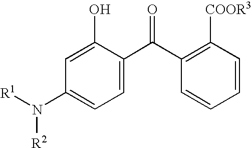
in which
R¹ and R², independently of one another are hydrogen, C₁-C₂₀-alkyl, C₃-C₁₀-cycloalkyl or C₃-C₁₀-cycloalkenyl, where the substituents R¹ and R², together with the nitrogen atom to which they are bonded, can form a 5-membered or 6-membered ring and
R³ is a C₁-C₂₀-alkyl radical.

A particularly advantageous hydroxybenzophenone for the purposes of the present invention is hexyl 2-(4′-(diethylamino)-2′-hydroxybenzoyl)benzoate, which is characterized by the chemical structural formula

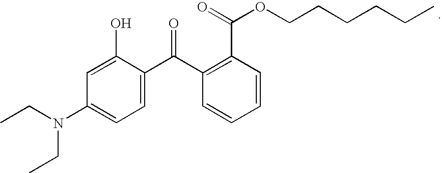

According to the invention, cosmetic or dermatological preparations comprise 0.1 to 20% by weight, advantageously 0.1 to 15% by weight, very particularly preferably 0.1 to 10% by weight, of one or more hydroxybenzophenones.

Suitable UV filters from the series of triazines which are advantageous according to the invention are, inter alia, the symmetrically substituted tris(2-ethylhexyl) 4,4′,4″-(1,3,5-triazine-2,4,6-triyltriimino)trisbenzoate, synonym: 2,4,6-tris[anilino[p-carbo-2′-ethyl-1′-hexyloxy)]-1,3,5-triazine [UVINUL T 150 (BASF)].

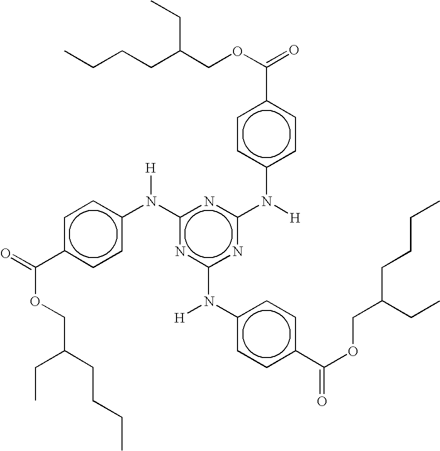

It is also possible to use asymmetrically substituted s-triazine derivatives, for example those as described in EP-A-570 838, the chemical structure of which is given by the generic formula 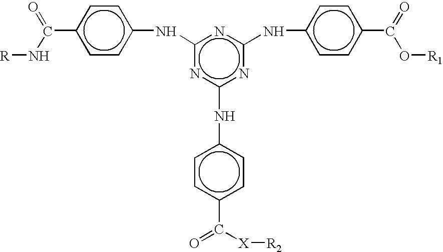
where
R is a branched or unbranched C₁-C₁₈-alkyl radical, a C₅-C₁₂-cycloalkyl radical, optionally substituted by one or more C₁-C₄-alkyl group,
X is an oxygen atom or an NH group,
R₁ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 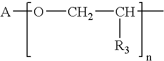
   in which
A is a branched or unbranched C₁-C₁₈-alkyl radical, C₅-C₁₂-cycloalkyl or aryl radical, optionally substituted by one or more C₁-C₄-alkyl groups,
R₃ is a hydrogen atom or a methyl group,
n is a number from 1 to 10,
R₂ is a branched or unbranched C₁-C₁₈-alkyl radical, a C₅-C₁₂-cycloalkyl radical, optionally substituted by one or more C₁-C₄-alkyl groups, if X is the NH group, and
a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 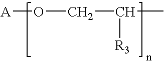
   in which
A is a branched or unbranched C₁-C₁₈-alkyl radical, a C₅-C₁₂-cycloalkyl or aryl radical, optionally substituted by one or more C₁-C₄-alkyl groups,
R₃ is a hydrogen atom or a methyl group,
n is a number from 1 to 10 if X is an oxygen atom.

A preferred, asymmetric s-triazine for the purposes of the present invention is characterized by the following structure 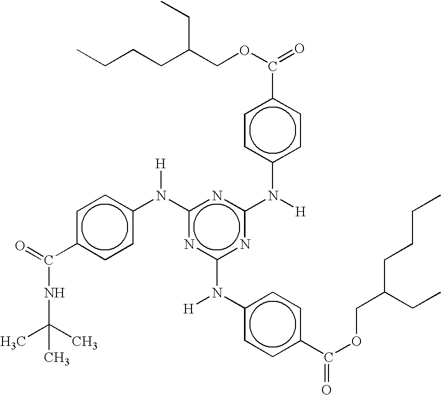
and is referred to as diethylhexylbutylamidotriazone (INCI: Diethylhexyl Butamido Triazone). It is available, inter alia, under the trade name UVASORB HEB from Sigma 3V.

Those asymmetrically substituted 5-triazines chosen from the group of substances which are described in EP-A-775 698 are particularly advantageous:

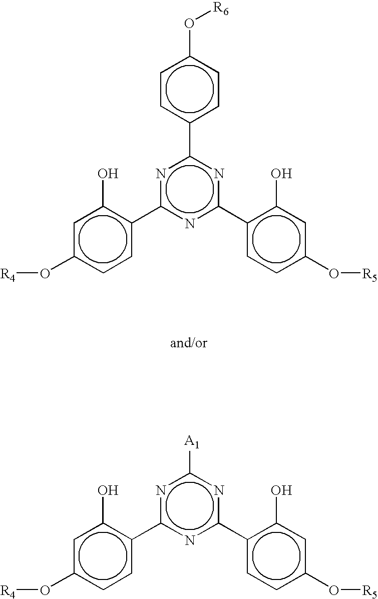

All of the bis-resorcinyltriazines mentioned in this specification, whether disclosed by generic formulae or by specific formulae, are advantageous for the purposes of the present invention. R₄ and R₅ are very particularly advantageously chosen from the group of branched and unbranched alkyl groups having 1 to 18 carbon atoms. The alkyl groups can also in turn advantageously be substituted by silyloxy groups.

A₁ is advantageously a substituted homo- or heterocyclic aromatic five-membered or six-membered ring.

The following compounds are very particularly advantageous: 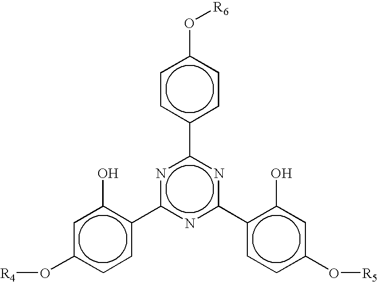
where R₆ is a hydrogen atom or a branched or unbranched alkyl group having 1 to 10 carbon atoms, in particular 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine or else Aniso Triazine), which is available under the trade name Tinosorb® S from CIBA-Chemikalien GmbH and is characterized by the following structure:

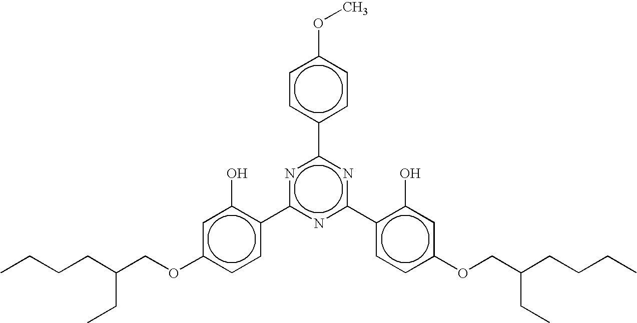

It is also in some instances advantageous to provide preparations according to the invention with a content of 2,4-bis {[4-(3-sulfonato)-2-hydroxypropyloxy)-2-hydroxy]phenyl}-6-(4-methoxyphenyl)-1,3,5-triazine sodium salt, which is characterized by the following structure:

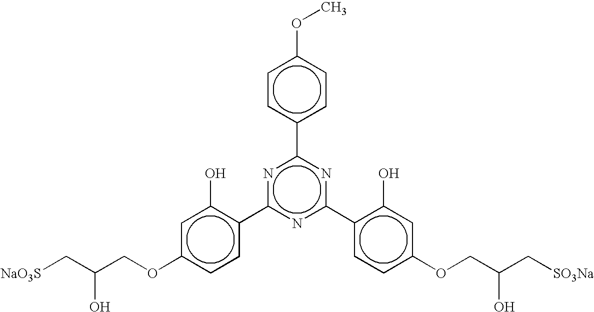

It is also advantageous to provide preparations according to the invention with 2,4-bis{[4-(3-(2-propyloxy)-2-hydroxypropyloxy)-2-hydroxy]phenyl}-6-(4-methoxyphenyl)-1,3,5-triazine, which is characterized by the following structure:

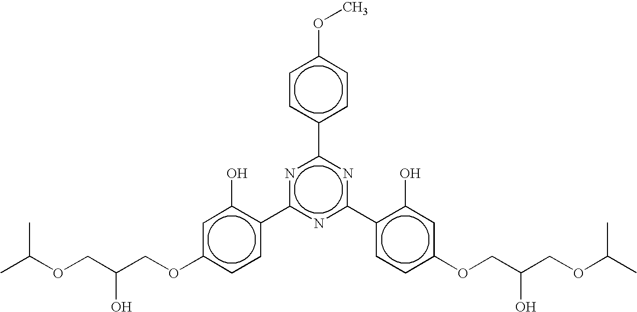

In addition, it is advantageous according to the invention to use 2,4-bis{[4-(2-ethylhexyloxy)-2-hydroxy]phenyl}-6-[4-(2-methoxyethylcarboxyl)phenylamino]-1,3,5-triazine as UV filter, which is characterized by the following structure:

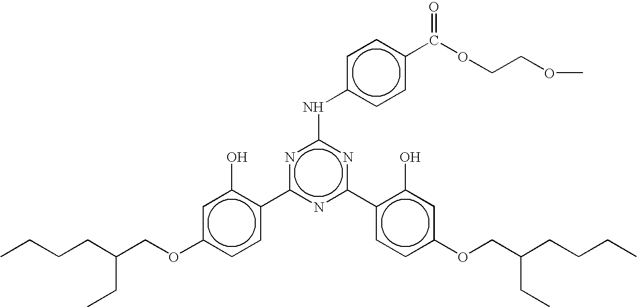

In addition 2,4-bis{[4-(3-(2-propyloxy)-2-hydroxypropyloxy)-2-hydroxy]phenyl}-6-[4-(ethylcarboxyl)phenylamino]-1,3,5-triazine is advantageous according to the invention. It is described by the following structure:

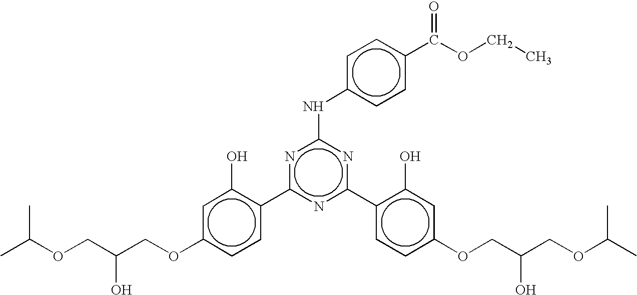

In addition 2,4-bis{[4-(2-ethylhexyloxy)-2-hydroxy]phenyl}-6-(1-methylpyrrol-2-yl)-1,3,5-triazine can be used for the purposes of the invention, which is characterized by the following structure:

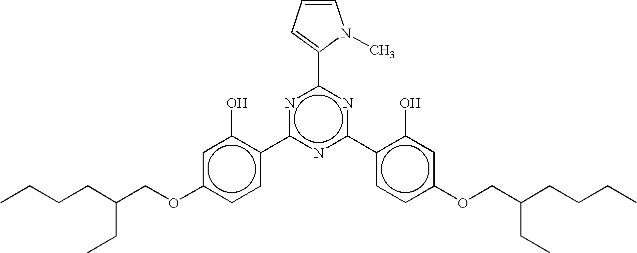

Further UV filters from the group of triazines which are advantageous for the purposes of the invention are 2,4-bis{[4-tris(trimethylsiloxysilylpropyloxy)-2-hydroxy]phenyl}-6-(4-methoxyphenyl)-1,3,5-triazine, which is characterized by the following structure, 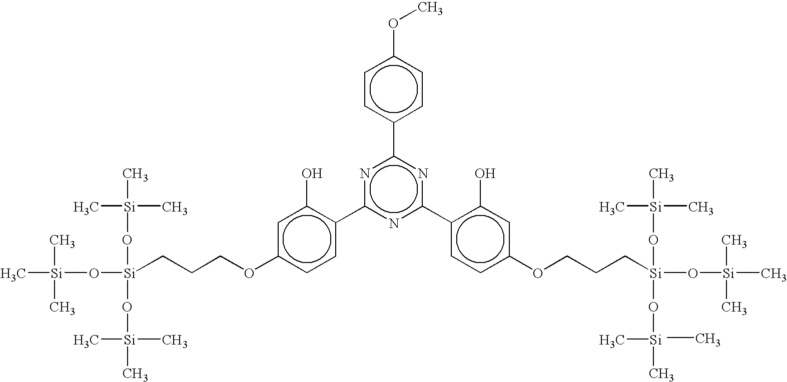
and to provide 2,4-bis {[4-(2-methylpropenyloxy)-2-hydroxy]phenyl}-6-(4-methoxyphenyl)-1,3,5-triazine, which is characterized by the following structure:

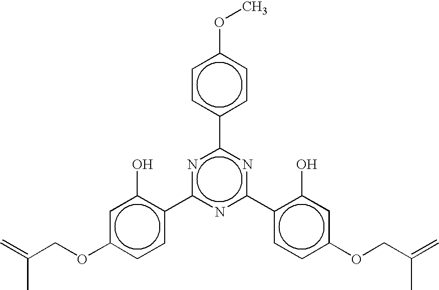

In addition, UV filters advantageous according to the invention include 2,4-bis{[4-(1′,1′,1′,3′,5═,5′,5′-heptamethylsiloxy-2-methylpropyloxy)-2-hydroxy]phenyl}-6-(4-methoxyphenyl)-1,3,5-triazine, which is characterized by the following structure:

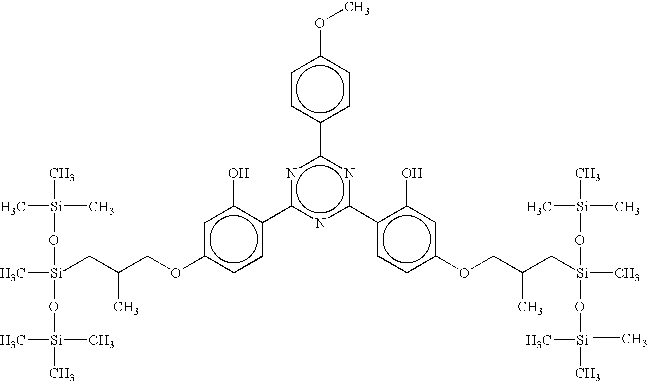

The triazine derivatives which are particularly preferred according to the invention are tris(2-ethylhexyl) 4,4′,4″-(1,3,5-triazine-2,4,6-triyltriimino)trisbenzoate, synonym: 2,4,6-tris[anilino(p-carbo-2′-ethyl-1′-hexyloxy)]-1,3,5-triazine [UVINUL T 150 (BASF)], diethylhexylbutylamidotriazone (INCI: Diethylhexyl Butamido Triazone, e.g. UVASORB HEB from Sigma 3V) and very particularly preferably 2,4-bis{[4-(2-ethylhexyloxy)-2-hydroxy]phenyl}-6-(4-methoxyphenyl)-1,3,5-triazine (INCI: bis-Ethylhexyloxyphenol Methoxyphenyl Triazine or Aniso Triazine, e.g. Tinosorb® S from CIBA).

Advantageous photoprotective filters for the purposes of the present invention which are characterized by the structural formula of benzotriazole are given by the structure

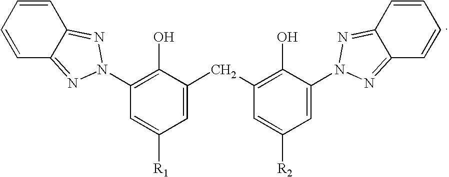

R₁ and R₂ can, independently of one another, be chosen from the group of branched or unbranched C₁-C₁₈-alkyl radicals which are optionally substituted by one or more C₁-C₄-alkyl groups, C₅-C₁₂-cycloalkyl radicals or aryl radicals.

The preferred benzotriazole derivative is 2,2′-methylenebis(6-(2H-benzotriazol-2-yl)-4-(1,1,3,3-tetramethylbutyl)phenol) [Tinosorb M (Ciba)], which is characterized by the chemical structural formula

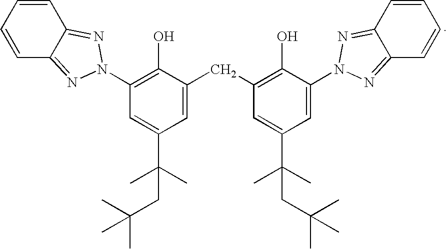

A further benzotriazole derivative preferred according to the invention is 2,2′-methylbis[6(2H-benzotriazol-2-yl)-4-(methyl)phenol] (MIXXIM BB/200 from Fairmount Chemical).

Further benzotriazole derivatives which are advantageous according to the invention are derived from the following structural formula (cf. WO 9522959):

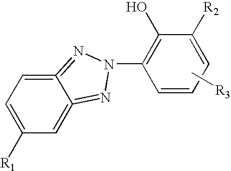

R₁ can be a hydrogen atom or an alkyl radical C₁-C₁₈. R₂ and R₃ may be identical or different. Particularly advantageous radicals for R₂ and R₃ are alkyl radicals from C₁ to C₁₈, phenyl radicals and optionally also silicone polymers.

Advantageous benzotriazoles for the purposes of the present invention are 2-(2′-hydroxy-3′,5′-di-t-amylphenyl)benzotriazole (CAS No.: 025973-551), 2-(2′-hydroxy-5′-octylphenyl)benzotriazole (CAS No.: 003147-75-9) or 2-(2′-hydroxy-5′-methylphenyl)benzotriazole (CAS No.: 2440-22-4).

A particularly advantageous broadband filter for the purposes of the present invention is also 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) [Mexoryl XL (Chimex)] with the INCI name Drometrizole Trisiloxane, which is characterized by the chemical structural formula

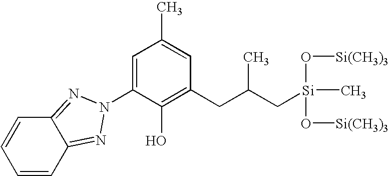

The total content of triazine derivatives and/or benzotriazole derivatives is chosen according to the invention from the range from 0.1 to 30% by weight, advantageously 0.1 to 20% by weight, particularly preferably 0.1 to 15% by weight, in each case based on the total weight of the preparation.

The cosmetic or dermatological photoprotective formulations according to the invention can have the customary composition and serve for cosmetic or dermatological sun protection, and also for the treatment, care and cleansing of the skin and/or of the hair and as a make-up product in decorative cosmetics.

Depending on their formulation, cosmetic or topical dermatological compositions for the purposes of the present invention can, for example, be used as skin protection cream, cleansing milk, day or night cream etc. It is in some cases possible and advantageous to use the compositions according to the invention as a base for pharmaceutical formulations.

For use, the cosmetic and dermatological preparations are applied to the skin and/or the hair in an adequate amount in the manner customary for cosmetics.

The cosmetic and dermatological preparations according to the invention can comprise cosmetic auxiliaries as are customarily used in such preparations, e.g. preservatives, preservative aids, bactericides, perfumes, substances for preventing foaming, dyes, pigments which have a coloring effect, thickeners, moisturizing and/or humectant substances, fillers, which improve the feel on the skin, fats, oils, waxes or other customary constitutents of a cosmetic or dermatological formulation, such as alcohols, polyols, polymers, foam stabilizers, electrolytes, organic solvents or silicone derivatives.

Advantageous preservatives for the purposes of the present invention are, for example, formaldehyde donors (such as, for example, DMDM hydantoin, which is available, for example, under the trade name Glydant™ from Lonza), iodopropyl butylcarbamates (e.g. those available under the trade names Glydant-2000, Glycacil-L, Glycacil-S from Lonza and/or Dekaben LMB from Jan Dekker), parabens (i.e. p-hydroxybenzoic alkyl esters, such as methyl-, ethyl-, propyl-, and/or butylparaben), phenoxyethanol, ethanol, benzoic acid and the like. Usually, according to the invention, the preservative system also advantageously comprises preservative assistants, such as, for example, ethylhexyloxyglycerol, glycine soya etc.

Particularly advantageous preparations are also obtained when antioxidants are used as additives or active ingredients. According to the invention, the preparations advantageously comprise one or more antioxidants. Favorable, but nevertheless optional, antioxidants which may be used are all antioxidants customary or suitable for cosmetic and/or dermatological applications.

For the purposes of the present invention, water-soluble antioxidants can be used particularly advantageously, such as, for example, vitamins, e.g. ascorbic acid and derivatives thereof.

Preferred antioxidants are also vitamin E and derivatives thereof, and vitamin A and derivatives thereof.

The amount of the antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or the antioxidants, it is advantageous to choose their respective concentrations from the range from 0.001 to 10% by weight, based on the total weight of the formulation.

If vitamin A or vitamin A derivatives, or carotenes or derivatives thereof, are the antioxidant or the antioxidants, it is advantageous to choose their respective concentrations from the range from 0.001 to 10% by weight, based on the total weight of the formulation.

It is particularly advantageous when the cosmetic preparations according to the present invention comprise cosmetic or dermatological active ingredients, preferred active ingredients being antioxidants which can protect the skin against oxidative stress.

Further advantageous active ingredients for the purposes of the present invention are natural active ingredients and/or derivatives thereof, such as, for example, alpha-lipoic acid, phytoene, D-biotin, coenzyme Q10, alpha-glucosylrutin, carnitine, carnosine, natural and/or synthetic isoflavonoids, creatine, taurine and/or β-alanine.

Formulations according to the invention which comprise, for example, known antiwrinkle active ingredients, such as flavone glycosides (in particular α-glycosylrutin), coenzyme Q10, vitamin E and/or derivative and the like are particularly advantageously suitable for the prophylaxis and treatment of cosmetic or dermatological changes in the skin, such as arise, for example, during skin aging (such as, for example, dryness, roughness and formation of dryness wrinkles, itching, reduced refatting (e.g. after washing), visible vascular dilations (telangiectases, cuperosis), flaccidity and formation of wrinkles and lines, local hyperpigmentation, hypopigmentation and incorrect pigmentation (e.g. age spots), increased susceptibility to mechanical stress (e.g. cracking) and the like). In addition, they are advantageously suitable against the appearance of dry or rough skin.

The water phase of the preparations according to the invention can advantageously comprise customary cosmetic auxiliaries, such as, for example, alcohols, in particular those of low carbon number, preferably ethanol and/or isopropanol, diols or polyols of low carbon number, and ethers thereof, preferably propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes, dihydroxyacetone and, in particular, one or more thickeners, which may advantageously be chosen from the group consisting of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of so-called carbopols, for example carbopol grades 980, 981, 1382, 2984, 5984, in each case individually or in combination.

Also advantageous are copolymers of C₁₀₋₃₀-alkyl acrylates and one or more monomers of acrylic acid, of methacrylic acid or esters thereof.

Compounds which bear the INCI name “Acrylates/C₁₀₋₃₀ Alkyl Acrylate Crosspolymer” are advantageous. Those available under the trade names Pemulen TR1 and Pemulen TR2 from B.F. Goodrich Company are particularly advantageous.

Compounds which bear the INCI name ammonium acryloyldimethyltaurate/vinylpyrrolidone copolymers are advantageous.

According to the invention, the ammonium acryloyldimethyltaurate/vinylpyrrolidone copolymer(s) advantageously have the empirical formula [C₇H₁₆N₂SO₄]ₙ[C₆H₉NO]ₘ, corresponding to a statistical structure as follows

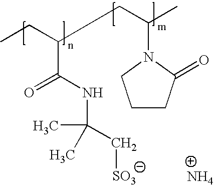

Preferred species for the purposes of the present invention are listed in the Chemical Abstracts under the registry numbers 58374-69-9, 13162-05-5 and 88-12-0 and are available under the trade name Aristoflex® AVC from Clariant GmbH.

Also advantageous are copolymers/crosspolymers comprising acryloyldimethyl taurates, such as, for example, Simugel® EG or Simugel® EG from Seppic S.A.

Further thickeners to be used advantageously according to the invention are also anionic polyurethanes which are dispersible or soluble in water. For the purposes of the present invention, polyurethane-1 and/or polyurethane-4, for example, are advantageous.

Particularly advantageous polyurethanes for the purposes of the present invention are the grades available under the trade name Avalure™ UR from B.F. Goodrich Company, such as, for example, Avalure™ UR 445, Avalure™ UR 450 and the like. Also advantageous for the purposes of the present invention is also the polyurethane available under the trade name Luviset Pur from BASF.

Moisturizers can also possibly be used. Moisturizers is the term used for substances or mixtures of substances which give cosmetic or dermatological preparations the property, following application and/or distribution on the surface of the skin, of reducing moisture release by the horny layer (also called transepidermal water loss, TEWL) and/or of positively influencing hydration of the horny layer.

Advantageous moisturizers for the purposes of the present invention are, for example, glycerol, lactic acid and/or lactates, in particular sodium lactate, butylene glycol, propylene glycol, biosaccharide gum-1, glycine soya, ethylhexyloxy glycerol, pyrrolidonecarboxylic acid and urea. In addition, it is particularly advantageous to use polymeric moisturizers from the group of water-soluble and/or water-swellable and/or water-gellable polysaccharides. Hyaluronic acid, chitosan and/or a fucose-rich polysaccharide, for example, which is filed in the Chemical Abstracts under the registry number 178463-23-5 and is available, for example, under the name Fucogel®1000 from SOLABIA S.A., are particularly advantageous.

The cosmetic or dermatological preparations according to the invention can also advantageously, but not obligatorily, comprise fillers which, for example, further improve the sensory and cosmetic properties of the formulations and, for example, bring about or enhance a velvety or silky feel on the skin. Advantageous fillers for the purposes of the present invention are starch and starch derivatives (such as, for example, tapioca starch, distarch phosphate, aluminum or sodium starch octenyl succinate and the like), pigments which have neither predominantly UV-filter effect nor coloring effect (such as, for example, boron nitride etc.) and/or Aerosils® (CAS No.: 7631-86-9).

The oil phase of the formulations according to the invention is advantageously chosen from the group of polar oils, for example from the group of lecithins and of fatty acid triglycerides, namely the triglycerol esters of saturated and/or unsaturated, branched and/or unbranched alkanecarboxylic acids with a chain length of from 8 to 24, in particular 12 to 18, carbon atoms. The fatty acid triglycerides can, for example, advantageously be chosen from the group of synthetic, semisynthetic and natural oils, such as, for example, cocoglyceride, olive oil, sunflower oil, soybean oil, peanut oil, rapeseed oil, almond oil, palm oil, coconut oil, castor oil, wheatgerm oil, grapeseed oil, thistle oil, evening primrose oil, macadamia nut oil and the like.

Also advantageous according to the invention are, for example, natural waxes of animal and vegetable origin, such as, for example, beeswax and other insect waxes and berry wax, shea butter and/or lanolin (wool wax).

Further advantageous polar oil components for the purposes of the present invention may also be chosen from the group of esters of saturated and/or unsaturated, branched and/or unbranched alkanecarboxylic acids with a chain length of from 3 to 30 carbon atoms and saturated and/or unsaturated, branched and/or unbranched alcohols with a chain length of from 3 to 30 carbon atoms, and also from the group of esters of aromatic carboxylic acids and saturated and/or unsaturated, branched and/or unbranched alcohols with a chain length of from 3 to 30 carbon atoms. Such ester oils can then advantageously be chosen from the group consisting of octyl palmitate, octyl cocoate, octyl isostearate, octyldodecyl myristate, octyldodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, and synthetic, semisynthetic and natural mixtures of such esters, such as, for example, jojoba oil.

In addition, the oil phase can advantageously be chosen from the group of dialkyl ethers and dialkyl carbonates, advantageous compounds being, for example, dicaprylyl ether (Cetiol OE) and/or dicaprylyl carbonate, for example that available under the trade name Cetiol CC from Cognis.

It is also preferred the oil component or the oil components from the group consisting of isoeicosane, neopentyl glycol diheptanoate, propylene glycol dicaprylate/dicaprate, caprylic/capric/diglyceryl succinate, butylene glycol dicaprylate/dicaprate, cocoglycerides (e.g. Myritol® 331 from Henkel), C₁₂₋₁₃-alkyl lactate, di-C₁₂₋₁₃-alkyl tartrate, triisostearine, dipentaerythrityl hexacaprylate/hexacaprate, propylene glycol monoisostearate, tricaprylin, dimethyl isosorbide. It is particularly advantageous when the oil phase of the formulations according to the invention has a content of C₁₂₋₁₅-alkyl benzoate or consists entirely of this.

Advantageous oil components are also, for example, butyloctyl salicylate (for example that available under trade name Hallbrite BHB from CP Hall), hexadecyl benzoate and butyloctyl benzoate and mixtures thereof (Hallstar AB) and/or diethylhexyl naphthalate (Hallbrite TQ from CP Hall or Corapan® TQ from Haarmann & Reimer).

Any desired mixtures of such oil and wax components can also be used advantageously for the purposes of the present invention.

The oil phase can also likewise advantageously also comprise nonpolar oils, for example those which are chosen from the group of branched and unbranched hydrocarbons and hydrocarbon waxes, in particular mineral oil, vaseline (petrolatum), paraffin oil, squalane, and squalene, polyolefins, hydrogenated polyisobutenes and isohexadecane. Among the polyolefins, polydecenes are the preferred substances.

The oil phase can advantageously also have a content of cyclic or linear silicone oils, or consist entirely of such oils, although it is preferred to use an additional content of other oil phase components apart from the silicone oil or the silicone oils.

Silicone oils are high molecular weight synthetic polymeric compounds in which silicon atoms are linked in a chain-like and/or reticular manner via oxygen atoms, and the remaining valencies of the silicon are saturated by hydrocarbon radicals (in most cases methyl, less often ethyl, propyl, phenyl groups etc.). Systematically, the silicone oils are referred to as polyorganosiloxanes. The methyl-substituted polyorganosiloxanes, which represent the most significant compounds in terms of number and are characterized by the following structural formula 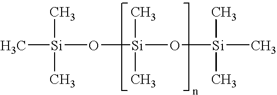
are also referred to as polydimethylsiloxane or Dimethicone (INCI). There are dimethicones with various chain lengths and with various molecular weights.

For the purposes of the present invention, particularly advantageous polyorganosiloxanes are, for example, dimethylpolysiloxanes [poly(dimethylsiloxane)], which are available, for example, under the trade names Abil 10 to 10 000 from Th. Goldschmidt. Also advantageous are phenylmethylpolysiloxanes (INCI: Phenyl Dimethicone, Phenyl Trimethicone), cyclic silicones (octamethylcyclotetrasiloxane or decamethylcyclopentasiloxane), which are also referred to as Cyclomethicones in accordance with INCI, amino-modified silicones (INCI: Amodimethicone) and silicone waxes, e.g. polysiloxane-polyalkylene copolymers (INCI: Stearyl Dimethicone and Cetyl Dimethicone) and dialkoxydimethylpolysiloxanes (Stearoxy Dimethicone and Behenoxy Stearyl Dimethicone), which are available as various Abil wax grades from Th. Goldschmidt. However, other silicone oils can also be used advantageously for the purposes of the present invention, for example cetyldimethicone, hexamethylcyclotrisiloxane, polydimethylsiloxane, poly(methylphenylsiloxane).

For the purposes of the present invention, it is also advantageous to create cosmetic and dermatological preparations whose main purpose is not protection against sunlight, but which nevertheless contain a content of further UV protection substances. Thus, for example, UV-A and/or UV-B filter substances are usually incorporated into day creams or make-up products. UV protection substances, like antioxidants and, if desired, preservatives, also represent effective protection of the preparations themselves against spoilage. Also favorable are cosmetic and dermatological preparations which are in the form of a sunscreen.

Accordingly, the preparations according to the invention can comprise further UV-A, UV-B and/or broadband filter substances. The formulations may, although do not have to, optionally also comprise one or more organic and/or inorganic pigments as UV filter substances, which may be present in the water phase and/or the oil phase.

In addition, the preparations according to the invention can also advantageously be in the form of so-called oil-free cosmetic or dermatological emulsions, which comprise a water phase and at least one UV filter substance liquid at room temperature as a further phase.

Particularly advantageous UV filter substances liquid at room temperature for the purposes of the present invention are homomenthyl salicylate (INCI: Homosalate), 2-ethylhexyl 2-cyano-3,3-diphenylacrylate (INCI: Octocrylene), 2-ethylhexyl 2-hydroxy-benzoate (2-ethylhexyl salicylate, octyl salicylate, INCI: Octyl Salicylate) and esters of cinnamic acid, preferably 2-ethylhexyl 4-methoxycinnamate (INCI: Octyl Methoxycinnamate) and isopentyl 4-methoxycinnamate (INCI: Isoamyl p-Methoxycinnamate).

Preferred inorganic pigments are metal oxides and/or other metal compounds which are insoluble or sparingly soluble in water, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides, and the sulfate of barium (BaSO₄).

For the purposes of the present invention, the pigments may advantageously also be used in the form of commercially available oily or aqueous predispersions. Dispersion auxiliaries and/or solubilization promoters may advantageously be added to these predispersions.

According to the invention, the pigments can advantageously be surface-treated (“coated”), the intention being, for example, to form or retain a hydrophilic, amphiphilic or hydrophobic character. This surface treatment can consist in providing the pigments with a thin hydrophilic and/or hydrophobic inorganic and/or organic layer by processes known per se. The various surface coatings may also comprise water for the purposes of the present invention.

Inorganic surface coatings for the purposes of the present invention may consist of aluminum oxide (Al₂O₃), aluminum hydroxide Al(OH)₃, or aluminum oxide hydrate (also: Alumina, CAS No.: 1333-84-2), sodium hexametaphosphate (NaPO₃)₆, sodium metaphosphate (NaPO₃)ₙ, silicon dioxide (SiO₂) (also: Silica, CAS No.: 7631-86-9), or iron oxide (Fe₂O₃). These inorganic surface coatings may be present on their own, in combination and/or in combination with organic coating materials.

Organic surface coatings for the purposes of the present invention may consist of vegetable or animal aluminum stearate, vegetable or animal stearic acid, lauric acid, dimethylpolysiloxane (also: Dimethicone), methylpolysiloxane (Methicone), simethicone (a mixture of dimethylpolysiloxane with an average chain length of from 200 to 350 dimethylsiloxane units and silica gel) or alginic acid. These organic surface coatings can be present on their own, in combination and/or in combination with inorganic coating materials.

Zinc oxide particles and predispersions of zinc oxide particles which are suitable according to the invention are available under the following trade names from the companies listed:

| Trade name | Coating | Manufacturer |
|---|---|---|
| Z-Cote HP1 | 2% Dimethicone | BASF |
| Z-Cote | / | BASF |
| ZnO NDM | 5% Dimethicone | Haarmann & Reimer |
| MZ-505S | 5% Methicone | Tayca Corporation |

Suitable titanium dioxide particles and predispersions of titanium dioxide particles are available under the following trade names from the companies listed:

| Trade name | Coating | Manufacturer |
|---|---|---|
| MT-100TV | Aluminum hydroxide/ | Tayca Corporation |
| | stearic acid | |
| MT-100Z | Aluminum hydroxide/ | Tayca Corporation |
| | stearic acid | |
| Eusolex T-2000 | Alumina/simethicone | Merck KgaA |
| Titanium dioxide T805 | Octyltrimethylsilane | Degussa |
| (Uvinul TiO₂) | | |
| MT-100AQ | Silica/aluminum hydroxide/ | Tayca Corporation |
| | alginic acid | |
| Eusolex T-Aqua | Water/alumina/sodium | Merck KgaA |
| | metaphosphate | |

Further advantageous UV-A filter substances for the purposes of the present invention are, inter alia, dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.

Advantageous further UV filter substances for the purposes of the present invention are sulfonated, water-soluble UV filters, such as, for example:
phenylene-1,4-bis(2-benzimidazyl)-3,3′-5,5′-tetrasulfonic acid and its salts, particularly the corresponding sodium, potassium or triethanolammonium salts, in particular the phenylene-1,4-bis(2-benzimidazyl)-3,3′,5,5′-tetrasulfonic acid bis-sodium salt with the INCI name Bisimidazylate (CAS No.: 180898-37-7), which is available, for example, under the trade name Neo Heliopan AP from Haarmann & Reimer;
salts of 2-phenylbenzimidazole-5-sulfonic acid, such as its sodium, potassium or its triethanolammonium salt, and the sulfonic acid itself with the INCI name Phenylbenzimidazole Sulfonic Acid (CAS No. 27503-81-7), which is available, for example, under the trade name Eusolex 232 from Merck or under Neo Heliopan Hydro from Haarrnann & Reimer;
1,4-di(2-oxo-10-sulfo-3-bomylidenemethyl)benzene (also: 3,3 ′-(1,4-phenylene-dimethylene)bis(7,7-dimethyl-2-oxobicyclo[2.2.1]hept-1-ylmethanesulfonic acid) and salts thereof (particularly the corresponding 10-sulfato compounds, in particular the corresponding sodium, potassium or triethanolammonium salt), which is also referred to as benzene-1,4-di(2-oxo-3-bomylidenemethyl-10-sulfonic acid). Benzene-1,4-di(2-oxo-3-bomylidenemethyl-10-sulfonic acid) has the INCI name Terephthalidene Dicamphor Sulfonic Acid (CAS No.: 90457-82-2) and is available, for example, under the trade name Mexoryl SX from Chimex;
sulfonic acid derivatives of 3-benzylidenecamphor, such as, for example, 4-(2-oxo-3-bornylidenemethyl)benzenesulfonic acid, 2-methyl-5-(2-oxo-3-bomylidenemethyl)-sulfonic acid and salts thereof.

Advantageous UV filter substances for the purposes of the present invention are also so-called broadband filters, i.e. filter substances which absorb both UV-A and also UV-B radiation.

An advantageous broadband filter for the purposes of the present invention is also 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane, which is available under the trade name Mexoryl® XL from Chimex.

The further UV filter substances may be oil-soluble or water-soluble.

Advantageous oil-soluble UV-B and/or broadband filter substances for the purposes of the present invention are, for example:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
derivatives of benzophenone, preferably 2-hydroxy-4-methoxybenzophenone, 2-hydroxy-4-methoxy-4′-methylbenzophenone, 2,2′-dihydroxy-4-methoxybenzo-phenone
and UV filters bonded to polymers.
3-(4-(2,2-bis(ethoxycarbonylvinyl)phenoxy)propenyl)methoxysiloxane/dimethyl-siloxane copolymer, which is available, for example, under the trade name Parsol® SLX from Hoffmann La Roche.

Advantageous water-soluble filter substances are, for example: sulfonic acid derivatives of 3-benzylidenecamphor, such as, for example, 4-(2-oxo-3-bomylidenemethyl)benzenesulfonic acid, 2-methyl-5-(2-oxo-3-bomylidenemethyl)-sulfonic acid and salts thereof.

A further photoprotective filter substance to be used advantageously according to the invention is ethylhexyl 2-cyano-3,3-diphenylacrylate (octocrylene), which is available from BASF under the name Uvinul® N 539.

Advantageous preparations for the purposes of the present invention which are characterized by high or very high UV-A and/or UV-B protection comprise, besides the filter substance or substances according to the invention, preferably also further UV-A and/or broadband filters, in particular dibenzoylmethane derivatives [for example 4-(tert-butyl)-4′-methoxydibenzoylmethane], phenylene-1,4-bis(2-benzimidazyl)-3,3′-5,5′-tetra-sulfonic acid and/or its salts, 1,4-di(2-oxo-10-sulfo-3-bomylidenemethyl)benzene and/or salts thereof, in each case individually or in any combinations with one another.

The list of said UV filters which can be used for the purposes of the present invention is not of course intended to be limiting.

The preparations according to the invention advantageously comprise the substances which absorb UV radiation in the UV-A and/or UV-B region in a total amount of, for example, 0.1% by weight to 30% by weight, preferably 0.1% by weight to 20% by weight, in particular 0.5% by weight to 15.0% by weight, in each case based on the total weight of the preparations, in order to provide cosmetic preparations which protect the hair and/or the skin against the entire range of ultraviolet radiation.

In addition, it may in some instances be advantageous to incorporate film formers into the cosmetic or dermatological preparations according to the invention, for example in order to improve the water resistance of the preparations, or to increase the UV protection performance (UV-A and/or UV-B boosting). Both water-soluble or dispersible and fat-soluble film formers are suitable, in each case individually or in combination with one another.

Advantageous water-soluble or dispersible film formers are, for example, polyurethanes (e.g. the Avalure® grades from Goodrich), dimethicone copolyol polyacrylate (Silsoft Surface® from Witco Organo Silicones Group), PVP/VA (VA=vinyl acetate) copolymer (Luviscol VA 64 Powder from BASF) etc.

Advantageous fat-soluble film formers are, for example, the film formers from the group of polymers based on polyvinylpyrrolidone (PVP)

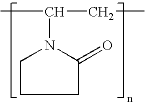

Particular preference is given to copolymers of polyvinylpyrrolidone, for example the PVP hexadecane copolymer and the PVP eicosene copolymer, which are available under the trade names Antaron V216 and Antaron V220 from GAF Chemicals Cooperation, and also tricontayl PVP and the like.

The present invention also provides for the use of hydrobenzophenones for increasing the solubility of triazine derivatives and benzotriazole derivatives in photoprotective cosmetic and/or dermatological preparations.

The invention further relates to the use of hydroxybenzophenones for increasing UV protection performance of triazine- and/or benzotriazole-containing photoprotective cosmetic and/or dermatological preparations.

Furthermore, the use of hydroxybenzophenones for increasing the stability of triazine- and/or benzotriazole-containing photoprotective cosmetic and/or dermatological preparations is provided by the present invention.

The examples below are intended to illustrate the present invention without limiting it. The numerical values in the examples mean percentages by weight, based on the total weight of the particular preparations. In the examples, “aminobenzophenone” is intended to be understood as meaning hexyl 2-(4′-(diethylamino)-2′-hydroxybenzoyl)benzoate.

### EXAMPLES

1. O/W sunscreen emulsions

| | 1 | 2 | 3 | 4 | 5 | 6 | 7 |
|---|---|---|---|---|---|---|---|
| Glycerol monostearate SE | 0.50 | 1.00 | | | | 1.50 | |
| Glyceryl stearate citrate | 3.00 | | | 1.00 | 2.00 | | 4.00 |
| Stearic acid | | 3.00 | 1.50 | 2.00 | | | |
| PEG-100 stearate | 0.50 | | 2.00 | | | 2.00 | |
| Cetyl phosphate | | | 0.35 | | 1.00 | | |
| Cetearyl sulfate | 0.50 | | | | | 0.75 | |
| Stearyl alcohol | | | 3.00 | | | 2.00 | 0.50 |
| Cetyl alcohol | 2.50 | 1.00 | | 1.50 | 0.50 | | 2.00 |
| Aminobenzophenone | 2.00 | 1.50 | 0.75 | 1.00 | 2.00 | 4.50 | 5.00 |
| Bis-ethylhexyloxyphenol | | 4.50 | | 2.00 | 2.50 | | 2.50 |
| methoxyphenyltriazine | | | | | | | |
| Ethylhexyltriazone | 4.00 | | 3.00 | 4.00 | | 2.00 | |
| Diethylhexylbutamidotriazone | 1.00 | | | 2.00 | 1.00 | | 1.00 |
| Methylenebisbenzotriazolyltetramethylbutylphenol | 2.00 | | 0.50 | 1.50 | 2.50 | | |
| Drometrizole trisiloxane | | | 0.50 | | | 1.00 | |
| Ethylhexyl methoxycinnamate | | | | 5.00 | 6.00 | | 8.00 |
| Butylmethoxydibenzoylmethane | | | 2.00 | | | 2.00 | 1.50 |
| Disodium phenyldibenzimidazoletetrasulfonate | 2.50 | | 0.50 | 2.00 | | | 0.30 |
| Octocrylene | | 4.00 | | | | | 7.50 |
| Phenylbenzimidazolesulfonic acid | 0.50 | | | 3.00 | | | |
| Ethylhexyl salicylate | | | 3.00 | | | | 5.00 |
| Terephthalidenedicamphorsulfonic | | 1.50 | | | 1.00 | 0.50 | |
| acid | | | | | | | |
| Diethylhexyl 2,6-naphthalate | | 4.00 | | | | | |
| Titanium dioxide MT-100Z | 1.00 | | | 3.00 | 2.00 | | 1.50 |
| Zinc oxide HP1 | | | | 0.25 | | 2.00 | |
| C12-15 alkyl benzoate | | 2.50 | | | 4.00 | 7.00 | |
| Dicaprylyl ether | | | 3.50 | | 2.00 | | |
| Butylene glycol dicaprylate/ | 5.00 | | | 6.00 | | | |
| dicaprate | | | | | | | |
| Dicaprylyl carbonate | | | 6.00 | | | 2.00 | |
| Dibutyl adipate | 2.50 | | 3.00 | | | | |
| Cocoglyceride | 4.50 | 7.50 | | | 3.00 | | |
| Dimethicone | | 0.50 | 1.00 | | 2.00 | | |
| Cyclomethicone | 7.00 | | | 5.50 | | | 0.50 |
| Shea butter | | 2.00 | | | | | |
| PVP hexadecane copolymer | 0.50 | | | 0.50 | 1.00 | | 1.00 |
| Glycerol | 3.00 | 7.50 | | 7.50 | 5.00 | | 2.50 |
| Xanthan gum | 0.15 | | 0.05 | | 0.35 | | 0.30 |
| Sodium carbomer | | 0.20 | 0.10 | 0.20 | | | |
| Vitamin E acetate | 0.50 | | 0.25 | | 0.75 | | 1.00 |
| Fucogel ® 1000 | | 3.50 | 10.00 | | | | |
| Glycine soya | | | | 0.50 | | 1.50 | 1.00 |
| Ethylhexyloxyglycerol | 0.35 | | | | | | 0.75 |
| DMDM hydantoin | | 0.60 | 0.40 | 0.20 | | | |
| Glycacil-L ® | | | | 0.18 | 0.20 | | |
| Methylparaben | 0.15 | | 0.25 | | 0.50 | | |
| Phenoxyethanol | 1.00 | 0.40 | | 0.40 | 0.50 | 0.40 | |
| EDTA | 0.02 | | 0.05 | | | | |
| Iminodisuccinic acid | | | | 0.25 | 1.0 | | |
| Ethanol | | 2.00 | 1.50 | | 3.00 | 4.50 | 5.00 |
| Perfume | 0.10 | 0.20 | 0.35 | | | 0.40 | 0.20 |
| Water | ad 100 | ad 100 | ad 100 | ad 100 | ad 100 | ad 100 | ad 100 |

| | | | | |
|---|---|---|---|---|
| 1 | 2 | 3 | 4 | 5 |

2. Hydrodispersions

| | | | | | |
|---|---|---|---|---|---|
| Ceteareth-20 | 1.00 | | | 0.5 | |
| Cetyl alcohol | | | 1.00 | | |
| Sodium carbomer | | 0.20 | | 0.30 | |
| Acrylates/C10-30 alkyl acrylate cross | 0.50 | | 0.40 | 0.10 | 0.50 |
| polymer | | | | | |
| Xanthan gum | | 0.30 | 0.15 | | 0.50 |
| Aminobenzophenone | 2.50 | 3.00 | 1.00 | 0.50 | 1.50 |
| Bisethylhexyloxyphenol | | 1.50 | | 2.00 | 2.50 |
| methoxyphenyltriazine | | | | | |
| Ethylhexyltriazone | 4.00 | | 3.00 | | 1.00 |
| Diethylhexylbutamidotriazone | 1.00 | | | 2.00 | |
| Drometrizole trisiloxane | | | 1.00 | | 1.50 |
| Methylenebisbenzotriazolyl | 2.50 | 0.50 | | | |
| tetramethylbutylphenol | | | | | |
| Ethylhexyl methoxycinnamate | | | | 5.00 | 8.00 |
| Butylmethoxydibenzoylmethane | | 0.50 | | 3.00 | 2.50 |
| Disodium phenyldibenzimidazoletetrasulfonate | 0.50 | | | | 3.00 |
| Octocrylene | | 4.00 | 3.90 | | 6.50 |
| Phenylbenzimidazolesulfonic acid | 0.50 | | | 3.00 | |
| Terephthalidenedicamphorsulfonic acid | | 0.50 | | | 1.00 |
| Parsol ® SLX | 5.00 | | | | 2.00 |
| Titanium dioxide MT-100TV | 0.50 | | 2.00 | | 1.00 |
| Zinc oxide HP1 | | | | | 3.00 |
| C12-15 alkyl benzoate | 2.00 | 2.50 | | | |
| Dicaprylyl ether | | 4.00 | | | |
| Butylene glycol dicaprylate/dicaprate | 4.00 | | 2.00 | 6.00 | |
| Dicaprylyl carbonate | | 2.00 | 6.00 | | |
| Dimethicone | | 0.50 | 1.00 | | |
| Phenyltrimethicone | 2.00 | | | 0.50 | |
| Shea butter | | 2.00 | | 5.00 | |
| PVP hexadecane copolymer | 0.50 | | | 0.50 | 1.00 |
| Tricontanyl PVP | 0.50 | | 1.00 | | |
| Ethylhexylglycerol | | | 1.00 | | 0.80 |
| Glycerol | 3.00 | 7.50 | | 7.50 | 8.50 |
| Glycine soya | | | 1.50 | | 1.00 |
| Vitamin E acetate | 0.50 | | 0.25 | | 1.00 |
| Alpha-glucosylrutin | | 0.60 | | | 0.25 |
| Fucogel ® 1000 | | 2.50 | 0.50 | | 2.00 |
| DMDM hydantoin | | 0.60 | 0.40 | 0.20 | |
| Glycacil-S ® | 0.20 | | | | |
| Methylparaben | 0.50 | | 0.25 | 0.15 | |
| Phenoxyethanol | 0.50 | 0.40 | | 1.00 | |
| Disodium EDTA | | 0.01 | 0.05 | | 0.10 |
| Ethanol | 3.00 | 2.00 | 1.50 | | 7.00 |
| Perfume | 0.20 | | 0.50 | 0.40 | |
| Water | ad 100 | ad 100 | ad 100 | ad 100 | ad 100 |

3. W/O sunscreen emulsions

| | | | | | |
|---|---|---|---|---|---|
| Cetyldimethicone copolyol | | 2.50 | | 4.00 | |
| Polyglyceryl-2 dipolyhydroxystearate | 5.00 | | | | 4.50 |
| PEG-30 dipolyhydroxystearate | | | 5.00 | | |
| Aminobenzophenone | 3.50 | 4.00 | 5.00 | 1.50 | 0.25 |
| Bisethylhexyloxyphenol | 2.00 | | | 2.00 | 2.50 |
| methoxyphenyltriazine | | | | | |
| Ethylhexyltriazone | | | 3.00 | 4.00 | |
| Diethylhexylbutamidotriazone | 1.00 | | | 2.00 | |
| Methylenebisbenzotriazolyltetramethylbutylphenol | | 3.50 | 2.00 | | |
| Drometrizole trisiloxane | | 1.00 | | | 1.50 |
| Ethylhexyl methoxycinnamate | | 8.00 | | 5.00 | 4.00 |
| Butylmethoxydibenzoylmethane | 1.00 | | | | 0.70 |
| Disodium phenyldibenzimidazoletetrasulfonate | | 1.00 | | 2.00 | 2.00 |
| Octocrylene | 10.00 | | 7.50 | | 2.50 |
| Phenylbenzimidazolesulfonic acid | 0.50 | | | 3.00 | 2.00 |
| Terephthalidenedicamphorsulfonic acid | | | 1.00 | | 0.50 |
| Titanium dioxide T805 | | 2.00 | | | 3.00 |
| Zinc oxide Z-Cote HP1 | 1.00 | | | 8.00 | 2.00 |
| Mineral oil | | 12.00 | 10.0 | | 8.00 |
| C12-15 alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone | | 4.00 | 1.00 | 5.00 | |
| Cyclomethicone | 2.00 | 25.00 | | | 2.00 |
| Shea butter | | | 3.00 | | |
| Vaseline | | 4.50 | | | |
| PVP hexadecane copolymer | 0.50 | | | 0.50 | 1.00 |
| Ethylhexylglycerol | | 0.30 | 1.00 | | 0.50 |
| Glycerol | 3.00 | 7.50 | | 7.50 | 8.50 |
| Glycine soya | | 1.00 | 1.50 | | 1.00 |
| MgSO₄ | 1.00 | 0.50 | | 0.50 | |
| MgCl₂ | | | 1.00 | | 0.70 |
| Vitamin E acetate | 0.50 | | 0.25 | | 1.00 |
| Ascorbyl palmitate | 0.50 | | | 2.50 | |
| Fucogel ® 1000 | | | | 3.50 | 7.50 |
| DMDM hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Iminodisuccinic acid | | | 0.12 | | |
| Ethanol | 3.00 | | 1.50 | | 5.00 |
| Perfume | 0.20 | | 0.40 | 0.35 | |
| Water | ad 100 | ad 100 | ad 100 | ad 100 | ad 100 |

4. Solid-stabilized emulsions

| | | | | | |
|---|---|---|---|---|---|
| Mineral oil | | | 16.0 | 16.0 | |
| Octyldodecanol | 9.0 | 9.0 | 5.0 | | |
| Caprylic/capric triglyceride | 9.0 | 9.0 | 6.0 | | |
| C12-15 alkyl benzoate | | | | 5.0 | 8.0 |
| Butylene glycol dicaprylate/dicaprate | | | | | 8.0 |
| Dicaprylyl ether | 9.0 | | | 4.0 | |
| Dicaprylyl carbonate | | 9.0 | | | |
| Hydroxyoctacosanyl hydroxystearate | 2.0 | 2.0 | 2.0 | 2.0 | 1.5 |
| Disteardimonium hectorite | 1.0 | 0.75 | | 0.5 | 0.25 |
| Cera microcristallina + paraffinum | | 0.35 | | | 5.0 |
| liquidum | | | | | |
| Hydroxypropylmethylcellulose | | | 0.1 | | 0.05 |
| Dimethicone | | | | | 3.0 |
| Aminobenzophenone | 3.0 | 5.0 | 2.0 | 5.5 | 0.75 |
| Diethylhexylbutamidotriazone | | 2.0 | | | 4.0 |
| Ethylhexyltriazone | 2.0 | | 1.5 | 4.0 | |
| Methylenebisbenzotriazolyltetramethylbutylphenol | 0.5 | | | 2.0 | 1.0 |
| Drometrizole trisiloxane | | 0.5 | | 1.0 | |
| Bis-ethylhexyloxyphenol | | | 3.0 | | 4.0 |
| methoxyphenyltriazine | | | | | |
| Butyl methoxydibenzoylmethane | | 0.5 | 3.50 | | 0.5 |
| Ethylhexyl methoxycinnamate | 6.0 | | | | 3.0 |
| Octocrylene | | 7.5 | 10.0 | | |
| Terephthalidenedicamphorsulfonic acid | | 1.0 | 0.5 | | 1.50 |
| Disodium phenyldibenzimidazoletetra- | 2.50 | | 3.1 | | |
| sulfonate | | | | | |
| Titanium dioxide + alumina + | | 2.0 | 4.0 | 2.0 | 4.0 |
| simethicone + aqua | | | | | |
| Titanium dioxide + trimethoxycaprylylsilane | 4.0 | | | | 3.0 |
| Zinc oxide Z-Cote HP1 | 2.5 | | | 6.0 | |
| Silica dimethylsilylate | | | 1.0 | | |
| Boron nitride | 2.0 | | | | |
| Starch/sodium metaphosphate polymer | | 0.5 | | | |
| Diethylhexyl 2,6-naphthalate | 5.0 | 7.0 | 8.5 | 3.0 | 4.5 |
| Tapioca starch | | | | 1.0 | |
| Sodium chloride | 1.0 | 1.0 | 1.0 | 1.0 | 1.0 |
| Glycerol | 5.0 | 10.0 | 3.0 | 6.0 | 10.0 |
| Trisodium EDTA | 1.0 | 1.0 | | 1.0 | |
| Methylparaben | | | | | 0.2 |
| Propylparaben | | | | | |
| Phenoxyethanol | | | 0.4 | 0.4 | 0.5 |
| Hexamidine diisethionate | | | | | 0.08 |
| Diazolidinylurea | | | 0.28 | 0.28 | |
| Alcohol | 5.0 | | | 2.5 | |
| Perfume | 0.25 | | 0.4 | 0.1 | |
| Water | ad 100 | ad 100 | ad 100 | ad 100 | ad 100 |

5. Sticks

| | 1 | 2 | 3 | 4 |
|---|---|---|---|---|
| Caprylic/capric triglyceride | 12 | 10 | 6 | |
| Octyldodecanol | 7 | 14 | 8 | 3 |
| Butylene glycol dicaprylate/ | | | | 12 |
| dicaprate | | | | |
| Pentaerythritol tetraisostearate | 10 | 6 | 8 | 7 |
| Polyglyceryl-3 diisostearate | 2.5 | | | |
| Bis-diglyceryl polyacyladipate-2 | 9 | 8 | 10 | 8 |
| Cetearyl alcohol | 8 | 11 | 9 | 7 |
| Myristyl myristate | 3.5 | 3 | 4 | 3 |
| Beeswax | 5 | 5 | 6 | 6 |
| Carnauba wax | 1.5 | 2 | 2 | 1.5 |
| Cera alba | 0.5 | 0.5 | 0.5 | 0.5 |
| C16-40 alkyl stearate | | 1.5 | 1.5 | 1.5 |
| Diethylhexyl 2,6-naphthalate | | | | 8.0 |
| Aminobenzophenone | 2 | 5.5 | 3 | 0.5 |
| Bis-ethylhexyloxyphenol | 2.5 | | | 1 |
| methoxyphenyltriazine | | | | |
| Ethylhexyltriazone | 2 | | | |
| Diethylhexylbutamidotriazone | | | | 3 |
| Drometrizole trisiloxane | 1 | | 4 | |
| Butyl methoxydibenzoylmethane | | 1 | 1 | |
| Z-Cote ® HP1 | | | | 4.5 |
| MT-100 TV | | 4 | 2.5 | |
| Titanium dioxide T 805 | | 3.6 | | 5 |
| Ethylhexyl methoxycinnamate | 3 | 3.6 | | 2.5 |
| Octocrylene | | | 7.5 | |
| Benzophenone-3 | | | 3.5 | |
| Tocopheryl acetate | 0.5 | 1 | | |
| Ascorbyl palmitate | 0.05 | | 0.05 | |
| Buxus Chinensis | 2 | 1 | | 1 |
| Perfume BHT | 0.1 | 0.25 | | 0.35 |
| Ricinus Communis | ad 100 | ad 100 | ad 100 | ad 100 |

6. PIT emulsions

| | 1 | 2 | 3 | 4 | 5 | 6 | 7 | 8 |
|---|---|---|---|---|---|---|---|---|
| Glycerol monostearate SE | 0.50 | 2.00 | 3.00 | 5.00 | | | 0.50 | 4.00 |
| Glyceryl isostearate | | | | | 3.50 | 4.00 | 2.00 | |
| Isoceteth-20 | | 0.50 | | | 2.00 | | | |
| Ceteareth-12 | | 5.00 | | 1.00 | | | | 3.50 |
| Ceteareth-20 | | | | 2.00 | | 2.50 | 3.00 | |
| PEG-100 stearate | 5.00 | | 1.00 | | 1.00 | | | 0.50 |
| Cetyl alcohol | 2.50 | 1.00 | | 1.50 | | 0.50 | 1.50 | |
| Cetyl palmitate | | | | 0.50 | | 1.00 | | |
| Cetyldimethicone copolyol | 0.50 | | | | 0.50 | | 1.00 | |
| Polyglyceryl-2 | | | | 0.75 | 0.25 | | | |
| dipolyhydroxystearate | | | | | | | | |
| Diethylhexyl 2,6-naphthalate | | 3.50 | | | | | | 4.50 |
| Aminobenzophenone | 2.00 | 2.00 | 4.00 | 1.50 | 3.00 | 3.00 | 0.75 | 2.50 |
| Bis-ethylhexyloxyphenol | | 0.75 | | 2.00 | | | 2.00 | |
| methoxyphenyltriazine | | | | | | | | |
| Drometrizole trisiloxane | | 1.00 | | | | 3.00 | | 1.00 |
| Dioctylbutamidotriazone | 0.50 | | | 3.00 | | 2.00 | | 1.50 |
| Ethylhexyltriazone | | | 2.00 | 4.00 | 2.50 | | 1.50 | 3.00 |
| Butylmethoxydibenzoylmethane | 1.50 | | 1.00 | | 5.00 | 1.00 | 0.75 | |
| Disodium phenyldibenzimidazoletetrasulfonate | | 2.00 | | | 1.00 | | | |
| Terephthalidenedicamphorsulfonic | | | 0.50 | | | | 1.00 | |
| acid | | | | | | | | |
| Ethylhexyl methoxycinnamate | 8.00 | | | 4.50 | 5.00 | 8.00 | | |
| Ethylhexyl salicylate | 4.00 | | | | 3.50 | 4.00 | | |
| Dimethicone diethylbenzalmalonate | | 4.50 | | | 3.50 | | | |
| Octocrylene | | | 5.00 | | 8.00 | 10.0 | | 7.50 |
| Phenylbenzimidazolesulfonic acid | 1.00 | 5.00 | | 3.00 | | | | |
| C12-15 alkyl benzoate | 3.50 | | | | 6.50 | | | |
| Cocoglycerides | | 3.00 | | 3.00 | | | | 3.50 |
| Dicaprylyl ether | 4.00 | | | | | | | |
| Butylene glycol dicaprylate/ | | 4.00 | | 3.00 | | | | |
| dicaprate | | | | | | | | |
| Dicaprylyl carbonate | | | | 0.50 | | | | 6.00 |
| Dibutyl adipate | | | 2.50 | | | | | 1.00 |
| Phenyltrimethicone | 2.00 | | | 3.00 | | | | |
| Cyclomethicone | | 3.00 | | | | | | 4.00 |
| Hydrogenated cocoglycerides | | | | 3.00 | 4.00 | | | 2.50 |
| Phenyltrimethicone | | | | | | 1.50 | 3.00 | |
| PVP hexadecene copolymer | | | | 1.00 | 1.50 | | | |
| Glycerol | 10.0 | 5.00 | | 7.50 | | 10.0 | | |
| Fucogel ® 1000 | | | 2.50 | 6.00 | | | | |
| Tocopherol | 1.00 | | | 0.75 | 0.50 | | 1.00 | |
| Shea butter | | 2.00 | 3.50 | | | | | 0.50 |
| Iodopropyl butylcarbamate | 0.12 | | | | 0.20 | | | |
| DMDM hydantoin | | | | 0.10 | | | | |
| Methylparaben | | 0.50 | 0.25 | | 0.45 | | | |
| Phenoxyethanol | 0.50 | 0.40 | | 1.00 | | | | 1.00 |
| Octoxyglycerol | | 0.30 | | | 1.00 | 0.35 | | |
| Ethanol | | | | 2.00 | | 6.00 | 7.50 | 4.00 |
| Trisodium EDTA | | 0.40 | | 0.15 | | 0.20 | | 0.50 |
| Perfume | 0.20 | | 0.20 | 0.20 | 0.45 | | | 0.20 |
| Water | ad 100 | ad 100 | ad 100 | ad 100 | ad 100 | ad 100 | ad 100 | ad 100 |

1-10. (canceled)

11. A photoprotective cosmetic or dermatological composition, wherein the composition comprises
(a) at least one hydroxybenzophenone; and
(b) at least one of a triazine derivative and a benzotriazole derivative.

12. The composition of claim 11, wherein the at least one hydroxybenzophenone comprises at least one hydroxybenzophenone of the formula: 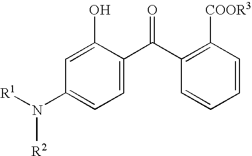
where R¹ and R², independently of one another, represent hydrogen, C₁-C₂₀-alkyl, C₃-C₁₀-cycloalkyl or C₃-C₁₀-cycloalkenyl or, together with the nitrogen atom to which they are bonded, R¹ and R² form a 5-membered or 6-membered heterocyclic ring; and R³ is a C₁-C₂₀-alkyl radical.

13. The composition of claim 12, wherein the at least one hydroxybenzophenone comprises hexyl 2-(4′-(diethylamino)-2′-hydroxybenzoyl)benzoate of the formula:

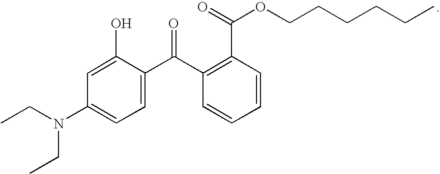

14. The composition of claim 11, wherein the at least one hydroxybenzophenone is present in a concentration of from 0.1% to 30% by weight, based on a total weight of the composition.

15. The composition of claim 14, wherein the concentration is from 0.1% to 15% by weight.

16. The composition of claim 15, wherein the concentration is up to 10% by weight.

17. The composition of claim 11, wherein the composition comprises at least one triazine derivative which is selected from tris(2-ethylhexyl) 4,4′,4″-(1,3,5-triazine-2,4,6-triyltriimino)trisbenzoate, diethylhexylbutylamidotriazone and 2,4-bis {[4-(2-ethylhexyloxy)-2-hydroxy]phenyl}-6-(4-methoxyphenyl)-1,3,5-triazine.

18. The composition of claim 17, wherein the composition comprises 2,4-bis{[4-(2-ethylhexyloxy)-2-hydroxy]phenyl}-6-(4-methoxyphenyl)-1,3,5-triazine.

19. The composition of claim 11, wherein the composition comprises at least one benzotriazole derivative which is selected from 2-(2H-benzotriazol-2-yl)-4-methyl-6-[2-methyl-3-[1,3,3,3-tetramethyl-1-[(trimethylsilyl)oxy]disiloxanyl]propyl]phenol and 2,2′-methylenebis(6-(2H-benzotriazol-2-yl)-4-(1,1,3,3-tetramethylbutyl)phenol).

20. The composition of claim 11, wherein the at least one of a triazine derivative and a benzotriazole derivative is present in a total concentration of from 0.1% to 30% by weight, based on a total weight of the composition.

21. The composition of claim 20, wherein the concentration is up to 20% by weight.

22. The composition of claim 21, wherein the concentration is up to 15% by weight.

23. The composition of claim 11, wherein the composition further comprises at least one flavone glycoside.

24. The composition of claim 23, wherein the composition comprises α-glucosylrutin.

25. The composition of claim 11, wherein the composition further comprises at least one of vitamin E and a derivative thereof.

26. An emulsion which comprises the composition of claim 11.

27. A hydrodispersion which comprises the composition of claim 11.

28. A stick which comprises the composition of claim 11.

29. The composition of claim 11, wherein the composition comprises from 0.1% to 15% by weight of the at least one hydroxybenzophenone and from 0.1% to 20% by weight of the at least one of a triazine derivative and a benzotriazole derivative.

30. The composition of claim 29, wherein the at least one hydroxybenzophenone comprises hexyl 2-(4′-(diethylamino)-2′-hydroxybenzoyl)benzoate.

31. The composition of claim 30, wherein the composition comprises at least one of tris(2-ethylhexyl) 4,4′,4″-(1,3,5-triazine-2,4,6-triyltriimino)trisbenzoate, diethylhexylbutylamido-triazone, 2,4-bis{[4-(2-ethylhexyloxy)-2-hydroxy]phenyl}-6-(4-methoxyphenyl)-1,3,5-triazine, 2-(2H-benzotriazol-2-yl)-4-methyl-6-[2-methyl-3-[1,3,3,3-tetramethyl-1-[(trimethylsilyl)oxy]-disiloxanyl]propyl]phenol and 2,2′-methylenebis(6-(2H-benzotriazol-2-yl)-4-(1,1,3,3-tetramethyl-butyl)phenol).

32. The composition of claim 31, wherein the composition comprises 2,4-bis{[4-(2-ethylhexyloxy)-2-hydroxy]phenyl}-6-(4-methoxyphenyl)-1,3,5-triazine.

33. The composition of claim 31, wherein the at least one hydroxybenzophenone is present in a concentration of up to 10% by weight.

34. The composition of claim 33, wherein the at least one of a triazine derivative and a benzotriazole derivative is present in a total concentration of up to 15% by weight, based on a total weight of the composition.

35. A method of increasing at least one of the solubility and the stability of a triazine derivative or a benzotriazole derivative in a photoprotective cosmetic or dermatological composition, wherein the method comprises incorporating into the composition at least one hydroxybenzophenone in an amount which increases at least one of the solubility and the stability of the triazine derivative or benzotriazole derivative.

36. The method of claim 35, wherein the at least one hydroxybenzophenone comprises hexyl 2-(4′-(diethylamino)-2′-hydroxybenzoyl)benzoate.

37. A method of increasing the UV protection performance of a triazine derivative or a benzotriazole derivative in a photoprotective cosmetic or dermatological composition, wherein the method comprises incorporating into the composition at least one hydroxybenzophenone in an amount which increases the UV protection performance of the triazine derivative or benzotriazole derivative.

38. The method of claim 37, wherein the at least one hydroxybenzophenone comprises hexyl 2-(4′-(diethylamino)-2′-hydroxybenzoyl)benzoate.
